(19) United States
(12) Patent Application Publication
(10) Pub. No.: US 2026/0174541 A1
(43) Pub. Date: Jun. 25, 2026

(54) STENT GRAFT SYSTEM AND METHOD OF IMPLANTATION

(71) Applicant: HANGZHOU INNOCARDIAC MEDTECH CO., LTD, Hangzhou (CN)

(72) Inventor: Yimin ZHAO, Hangzhou (CN)

(21) Appl. No.: 19/430,376

(22) Filed: Dec. 23, 2025

Related U.S. Application Data
(63) Continuation-in-part of application No. PCT/CN2025/093388, filed on May 8, 2025.
(60) Provisional application No. 63/738,495, filed on Dec. 23, 2024.

(30) Foreign Application Priority Data
Apr. 15, 2025 (CN) 202510465179.6

(51) Int. Cl. A61F 2/07; A61F 2/06

(52) U.S. Cl. CPC A61F 2/07; A61F 2002/061; A61F 2002/065; A61F 2250/0098

(57) ABSTRACT

This invention provides a method for implanting a stent graft system into a vessel, said vessel comprises a trunk, a positioning branch vessel, and a first branch vessel, said method comprises the steps of: a) providing said stent graft system; b) delivering said stent graft into said trunk; c) aligning said positioning assembly with said positioning branch vessel; d) releasing said stent graft, such that said stent graft conforms to inner wall of said trunk and said positioning assembly aligns with the root of said positioning branch vessel; e) delivering said first branch vessel stent graft into said first branch vessel via said first branch vessel fenestration assembly (11); f) releasing said first branch vessel stent graft, such that said second opening aligns with the root of said first branch vessel, said first sealing zone (111) forms a sealed connection with said first branch vessel stent graft.

### FIELD OF THE INVENTION

The present invention relates to fenestrated stent grafts, particularly those stent grafts for the treatment of vascular diseases involving both the trunk and branch vessels.

### BACKGROUND OF THE INVENTION

The present invention is applicable for the treatment of vascular diseases involving both the trunk and branch vessels. For ease of explanation, an aortic dissection involving the aortic arch and supra-arch branches will be used as an example in the following description.

As shown in FIGS. 1A and 1B, the aortic arch region is composed of the ascending aorta (31), the innominate artery (32), the left common carotid artery (33), the left subclavian artery (34), and the descending aorta (35). Aortic dissection is a disease characterized by disruption of the medial layer of the aortic wall caused by hemorrhage within the vessel wall, resulting in separation of the vessel wall and subsequently forming a true lumen (36) and a false lumen (37), which may or may not communicate with each other. In most cases, aortic dissection originates from an intimal tear, through which blood enters the medial layer via an intimal tear opening (38), ultimately leading to rupture of the aorta or re-entry into the true lumen through another intimal tear opening. Based on the affected location, aortic dissection is classified into Stanford type A dissection (involving the ascending aorta), Stanford type B dissection (involving only the descending aorta), and non-A non-B dissection (involving the aortic arch without involving the ascending aorta).

In China, the prevalence of aortic dissection is 0.07% [1]. Among newly diagnosed cases of aortic dissection each year, Stanford type A dissections account for approximately 60%, while non-A non-B dissections account for approximately 10%.

For Stanford type A dissections and non-A non-B dissections, since the lesions involve the ascending aorta and the three supra-aortic branch vessels, and because the structural relationships between the trunk and branch vessels in this region, including the positions of branch vessel openings, the distances between branch vessels, and the orientations of branch vessel openings, vary among patients, there has been no standardized interventional stent product suitable for lesions involving two or more branch vessels and accommodating such variations. Therefore, conventional treatment still relies on open-chest surgery under cardiopulmonary bypass. However, surgical procedures present the following problems:
1) The surgical procedure is complex, lengthy, and difficult to disseminate: As shown in FIG. 2, the procedure must be performed under cardiopulmonary bypass, the surgeon dissects and excises the entire aortic arch and replaces it with an artificial graft (4), followed by completing at least five end-to-end anastomoses between the artificial graft and the native vessels (at the ascending aorta 41, the innominate artery 42, the left common carotid artery 43, the left subclavian artery 44, and the descending aorta 45). The surgery typically lasts approximately 6-10 hours, with an average cardiopulmonary bypass time of 2-3 hours, an average aortic cross-clamp time of 1.5-2 hours, and an average deep hypothermic circulatory arrest time of 20-30 minutes. In summary, the procedure is complex, highly time-consuming, and associated with a long learning curve for surgeons, making it difficult to disseminate.
2) The prolonged duration of deep hypothermic circulatory arrest increases the risk of ischemia in organs and lower limbs: Deep hypothermic circulatory arrest typically lasts 20-30 minutes, the cessation of circulation can easily cause ischemic and hypoxic injury to various organs, while deep hypothermia may further lead to pathophysiological alterations within the organs.
3) The above-mentioned end-to-end anastomosis at each site is technically demanding, and the anastomotic stomas are prone to bleeding. In addition, the mobilized serosal junctions of the native aortic arch tend to ooze significantly. These factors contribute to difficulty in achieving hemostasis.
4) The surgical risk is high: the operative mortality rate ranges from 10% to 33% outside China [3]. In China, the operative mortality rate ranges from 3.1% to 15.5%, with an incidence of acute respiratory insufficiency of 5%-15%, neurologic complications of 4%-30%, renal failure of 5%-12%, and postoperative hospital-acquired infection of approximately 12% [4]. The onset and progression of the disease are extremely rapid (the mortality rate of Stanford type A dissection within 48 hours after onset is 40%-50%), and since the patient transfer times are often prolonged, many patients die before receiving medical treatment.

With the global increase in the prevalence of hypertension, the incidence of the above diseases has risen correspondingly. Due to the characteristics described above, many patients are unable to obtain timely treatment locally and lose their lives, making these conditions a longstanding challenge for clinicians. Over the past decade, physicians worldwide have repeatedly attempted to use endovascular interventional repair to reduce the clinical difficulties described above. The main approaches include the following:
1) In-situ fenestration technique: A straight thoracic aortic stent graft is delivered via catheter into the aortic arch region. At the locations corresponding to the three supra-aortic branch vessels, the graft of the thoracic aortic stent graft is intraoperatively fenestrated in situ, and the fenestration openings are then expanded with a balloon. Through each expanded fenestration, a corresponding branch vessel stent graft is delivered sequentially via a catheter. This technique presents the following problems: a. Before fenestration, after implantation of the thoracic aortic stent graft, the openings of the corresponding branch vessels are isolated by the thoracic aortic stent graft and subjected to ischemia. If the ischemic duration is prolonged, extremely severe cerebral complications may occur. b. The fenestrations created in the graft are formed by physical tearing. After implantation of the branch vessel stent grafts, the branch vessel stents generally do not seal tightly against the torn fenestration edges, resulting in a high incidence of endoleak (blood flow escaping through the gap between the torn fenestration on the thoracic aortic stent graft and the branch vessel stent graft). The false lumen may continue to enlarge, and patients often require additional surgical interventions. c. The procedure is complex, time-consuming, and difficult to generalize. d. Fenestration creation and branch vessel stent implantation are typically performed via arteries in the neck or right arm (such as the brachial artery), using the supra-aortic branch vessels as access routes (referred to in interventional practice as “upper access”). Instruments repeatedly pass through these branch vessels, which commonly contain atherosclerotic plaques, increasing the risk of plaque dislodgement and intraoperative embolic stroke, or causing intimal injury that leads to the formation of new plaques in these branch vessels, which may subsequently detach and result in embolic stroke.
2) Inner-tunnel and modular bridging techniques: A thoracic aortic stent graft containing three inner tunnels is delivered via catheter into the aortic arch region, using the three inner tunnels as interfaces for bridging between the thoracic aortic stent graft and the branch vessel stent grafts. Among them, the inner tunnels corresponding to the innominate artery and the left common carotid artery extend from the roots of the respective branch vessel toward the ascending aorta, while the inner tunnel corresponding to the left subclavian artery extends from the root of the left subclavian artery toward the descending aorta. The catheter is inserted sequentially to deliver the branch vessel stent grafts corresponding to the innominate artery and the left common carotid artery via the upper access, and to deliver the branch vessel stent graft corresponding to the left subclavian artery via the femoral-artery access route (referred to in interventional practice as “lower access”). This technique presents similar problems: since the branch vessel stent grafts for the innominate artery and the left common carotid artery are implanted via upper access, the procedure is long and complex. Many operative maneuvers are performed within the supra-aortic branch vessels, increasing the risk of dislodging pre-existing plaques in the native branch vessels and causing intraoperative embolic stroke, or causing intimal injury that leads to new plaque formation and subsequent embolic stroke.
3) Hybrid surgery: Hybrid surgery generally consists of two steps. First, a surgical bypass is performed in the neck using an artificial graft to establish a connection between the innominate artery and the left common carotid artery, and between the left common carotid artery and the left subclavian artery. Second, a branched interventional stent graft containing an innominate artery stent is delivered by the catheter via lower access and implanted into the innominate artery of the aortic arch region, thereby restoring blood flow among the branch vessels in this region. This technique requires two separate stages, is surgically complex, and involves long waiting times for patients. Cervical bypass surgery may lead to complications such as bleeding and infection. Moreover, this mode of reconstruction significantly alters the native hemodynamics of the supra-aortic branches, and the bypass grafts may subsequently develop stenosis or occlusion.

All of the above methods suffer from the disadvantages of prolonged operative time, procedural complexity, a wide range of potential complications, a lack of standardized instruments and surgical techniques, and limited feasibility for widespread adoption in most hospitals.

### SUMMARY OF THE INVENTION

In one embodiment, this invention provides method for implanting a stent graft system into a vessel, said vessel comprises a trunk, a positioning branch vessel, and a first branch vessel, wherein said stent graft system comprises: a) a stent graft, comprising: i) a positioning assembly; ii) a first branch vessel fenestration assembly (11), said first branch vessel fenestration assembly (11) comprises a first sealing zone (111) and a first adjustment zone (112); said first sealing zone (111) comprises a third opening (1112); said first adjustment zone (112) comprises a first opening (1121), a second opening (1122), a first reinforcement structure (113), and a second reinforcement structure (114); a first branch vessel stent graft, said first branch vessel stent graft is configured to be released within said first branch vessel fenestration assembly (11); said first branch vessel stent graft comprises a first connecting anchoring segment, the diameter of said first connecting anchoring segment after being released is greater than said first opening (1121) of said first adjustment zone, such that said first connecting anchoring segment is retained at the cardiac-proximal end of said first opening (1121) of said first adjustment zone; said method comprises the steps of: a) providing said stent graft system; b) delivering said stent graft into said trunk; c) aligning said positioning assembly with said positioning branch vessel; d) releasing said stent graft, such that said stent graft conforms to inner wall of said trunk and said positioning assembly aligns with the root of said positioning branch vessel; e) delivering said first branch vessel stent graft into said first branch vessel via said first branch vessel fenestration assembly (11); f) releasing said first branch vessel stent graft, such that said second opening aligns with the root of said first branch vessel, said first sealing zone (111) forms a sealed connection with said first branch vessel stent graft.

### BRIEF DESCRIPTION OF THE FIGURES

FIG. 1A illustrates a schematic view of a normal aorta. FIG. 1B illustrates a schematic view of an aortic dissection.

FIG. 2 illustrates five end-to-end anastomoses between the artificial graft and the native vessels during open surgery.

FIG. 3 is a front view of the stent graft with a fenestration assembly.

FIG. 4 is a front view of the fenestration assembly.

FIG. 5 is a top view of the fenestration assembly.

FIG. 6 is a front view of the fenestration assembly.

FIG. 7 is a cross-sectional view of the first reinforcement structure (or second reinforcement structure) and the first radiopaque structure (or second radiopaque structure).

FIG. 8 shows the fenestration assembly (11) during the deployment of the fenestrated stent graft (1), aligned with the root and the center of the left common carotid artery, prior to the release and expansion of the fenestrated stent graft (1).

FIG. 9 illustrates a schematic view of the fenestrated stent graft (1) after release and expansion.

FIG. 10 illustrates the delivery of the branch vessel stent graft mounted on the delivery system to the left common carotid artery.

FIG. 11 illustrates a schematic view of the branch vessel stent graft for the left common carotid artery after release and expansion.

FIG. 12 illustrates the delivery of the branch vessel stent graft mounted on the delivery system to the innominate artery, at which time the second opening remains misaligned with the root of the branch vessel.

FIG. 13A illustrates a schematic view of the misalignment between the second opening and the root of the branch vessel.

FIG. 13B illustrates a schematic view of the alignment between the second opening and the root of the branch vessel.

FIG. 14 illustrates the branch vessel stent graft for the innominate artery after release and expansion, at which time the second opening is aligned with the root of the branch vessel.

FIG. 15 illustrates the branch vessel stent graft for the left subclavian artery after release and expansion, at which time the second opening is aligned with the root of the branch vessel.

FIG. 16 illustrates preoperative and postoperative clinical CT angiography for clinical trial cases 001-005.

FIG. 17 illustrates preoperative and postoperative clinical CT angiography for clinical trial cases 006-010.

FIG. 18 illustrates a comparison of velocity contour plots and streamline diagrams in a finite element analysis between the models with and without a sealing zone.

FIG. 19 illustrates a comparison of velocity contour plots in a finite element analysis between the models with and without a sealing zone.

FIG. 20 shows the main stent graft in an embodiment of this invention.

FIG. 21 shows the branch vessel stent graft in an embodiment of this invention; cylindrical

FIG. 22 shows the straight stent graft in an embodiment of this invention.

FIG. 23 shows the main stent graft delivery system in an embodiment of this invention.

FIG. 24 shows the branch vessel stent graft delivery system in an embodiment of this invention.

FIG. 25 shows the straight stent graft delivery system in an embodiment of this invention.

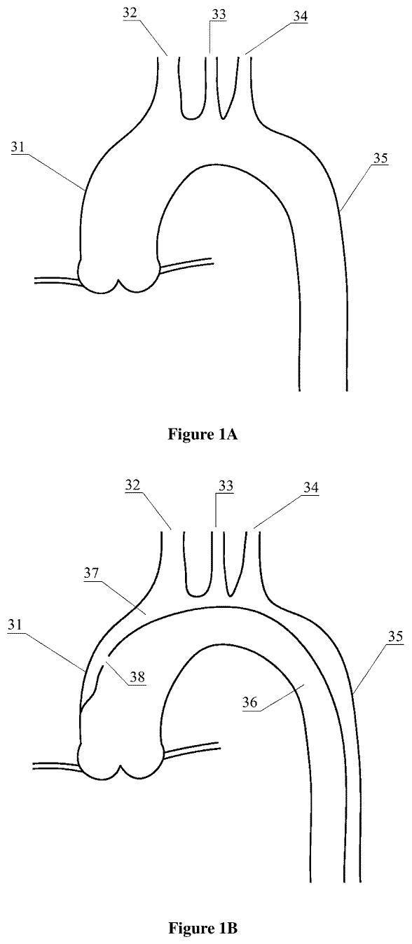

### DETAILED DESCRIPTION OF THE INVENTION

Vascular diseases involving both the trunk and the branch vessels are among the most life-threatening cardiovascular conditions, posing an extreme danger to patients and requiring emergency transfer to major cardiac centers for highly invasive open surgery. Many patients die because timely treatment is not available. Even among those who receive prompt surgical intervention, the operative mortality rate remains as high as 10-33%, and postoperative complications occur at rates of 4-30%. This has long been a major challenge in the clinical management of such patients. The principal advantageous effects of the present invention are as follows:
1) The present invention can replace most of the current open-chest or open-abdominal procedures requiring cardiopulmonary bypass and deep hypothermic circulatory arrest. Instead, the related diseases can be treated solely through catheter-based endovascular implantation, greatly reducing surgical trauma and lowering the high operative mortality and complication rates associated with open surgery, thereby providing an entirely new therapeutic approach for treating vascular diseases involving branch vessels;
2) The various innovations of the present invention allow all devices of this invention to be implanted through the lower-limb arteries. The procedure is simple, easy to learn, and readily adoptable by the majority of mid-level hospitals. This fundamentally changes the current situation in which such patients must be urgently transferred hundreds of kilometers to large hospitals for open surgery. As a result, many patients are able to receive timely and rapid treatment at nearby hospitals, significantly improving survival rates;
3) Compared with open surgical treatment, catheter-based interventional treatment of vascular diseases involving branch vessels is significantly less costly, and therefore offers substantial clinical and societal value.

For the treatment of vascular diseases involving both the trunk and the branch vessels, the present invention provides a stent graft with a fenestration assembly. In combination with the branch vessel stent grafts, this stent graft can reconstruct the diseased vessels. For example, in patients with Stanford type A or non-A non-B aortic dissection (including aneurysms, intramural hematomas, and multiple penetrating atherosclerotic ulcers in this region), the diseased aorta can be reconstructed using the stent graft provided by the present invention.

This invention provides a stent graft system. In one embodiment, said stent graft system comprises a) a stent graft, comprising: i) a positioning assembly; ii) a first branch vessel fenestration assembly (11), said first branch vessel fenestration assembly (11) comprises a first sealing zone (111) and a first adjustment zone (112); said first sealing zone (111) comprises a third opening (1112); said first adjustment zone (112) comprises a first opening (1121), a second opening (1122), a first reinforcement structure (113), and a second reinforcement structure (114); b) a first branch vessel stent graft, said first branch vessel stent graft is configured to be released within said first branch vessel fenestration assembly (11); wherein: said first reinforcement structure (113) maintains said first opening (1121), and said second reinforcement structure (114) maintains said second opening (1122); said first adjustment zone (112) is located between said first reinforcement structure (113) and said second reinforcement structure (114); said first sealing zone (111) extends from said second reinforcement structure (114) to said third opening (1112), such that said first branch vessel stent graft forms a planar sealed connection with said first sealing zone (111) when said first branch vessel stent graft is released and expanded; and said first branch vessel stent graft comprises a first connecting anchoring segment, the diameter of said first connecting anchoring segment after being released is greater than said first opening (1121) of said first adjustment zone, such that said first connecting anchoring segment is retained at the cardiac-proximal end of said first opening (1121) of said first adjustment zone.

In one embodiment, said sealing zone (111) comprises an extension from said second reinforcement structure to said third opening (1112), said extension begins from said second reinforcement structure towards said adjustment zone (112).

In one embodiment, said sealing zone (111) comprises an extension from said second reinforcement structure to said third opening (1112), said extension begins from said second reinforcement structure away from said adjustment zone (112).

In one embodiment, said sealing zone (111) comprises an extension from said second reinforcement structure to said third opening (1112), said extension begins from said second reinforcement structure simultaneously towards said adjustment zone (112) and away from said adjustment zone (112).

In one embodiment, said first reinforcement structure (113) comprises a first radiopaque structure (115); said second reinforcement structure (114) comprises a second radiopaque structure (116); or said first adjustment zone (112) comprises a third radiopaque structure (117).

In one embodiment, said positioning assembly comprises a positioning sealing zone (111) and a positioning adjustment zone (112); said positioning sealing zone (111) comprises a positioning third opening (1112); said positioning adjustment zone (112) comprises a positioning first opening (1121), a positioning second opening (1122), a positioning first reinforcement structure (113), and a positioning second reinforcement structure (114).

In one embodiment, said stent graft further comprises a positioning branch vessel stent graft, said positioning branch vessel stent graft is configured to be implanted into said positioning assembly.

In one embodiment, said first connecting anchoring segment comprises a flange stent and a flange graft.

In one embodiment, the relative sizes of said first opening (1121), said second opening (1122), and said third opening (1112) are selected from one or more of the following: a) said first opening (1121) is greater than said second opening (1122); b) said second opening (1122) is greater than said third opening (1112); c) said first opening (1121) is equal to said second opening (1122); d) said second opening (1122) is equal to said third opening (1112).

In one embodiment, said first adjustment zone (112) or said first sealing zone (111) is formed from a flexible cylindrical or frustoconical graft.

In one embodiment, said first opening (1121), said second opening (1122), or said third opening (1112) is circular or substantially circular in shape.

In one embodiment, said first reinforcement structure (113) or said second reinforcement structure (114) is made of superelastic materials (metals, alloys, polymers etc.).

In one embodiment, the connection between said first sealing zone (111) and said first branch vessel stent graft is a planar connection.

In one embodiment, said stent graft system further comprises a second branch vessel fenestration assembly and a second branch vessel stent graft; said second branch vessel fenestration assembly comprises a second sealing zone (111) and a second adjustment zone (112); said second sealing zone (111) comprises a third opening (1112); said second adjustment zone comprises a first opening (1121), a second opening (1122), a first reinforcement structure (113), and a second reinforcement structure (114); said second branch vessel stent graft comprises a second connecting anchoring segment, the diameter of said second connecting anchoring segment after being released is greater than said first opening (1121) of said second adjustment zone, such that said second connecting anchoring segment is retained at the cardiac-proximal end of said first opening (1121) of said second adjustment zone; said positioning assembly is configured to be implanted into the left common carotid artery; said first branch vessel fenestration assembly is configured to be implanted into the innominate artery; said second branch vessel fenestration assembly is configured to be implanted into the left subclavian artery.

This invention further provides a method for implanting a stent graft into a vessel, said vessel comprises a trunk, a positioning branch vessel, and a first branch vessel. In one embodiment, said method comprises the steps of: a) providing said stent graft system of this invention; b) delivering said stent graft into said trunk of said vessel; c) aligning said positioning assembly with said positioning branch vessel; d) releasing said stent graft, such that said stent graft conforms to the inner wall of said trunk and said positioning assembly aligns with the root of said positioning branch vessel; e) delivering said first branch vessel stent graft into said first branch vessel via said first branch vessel fenestration assembly (11); f) releasing said first branch vessel stent graft, such that said second opening aligns with the root of said first branch vessel, said first sealing zone (111) forms a planar sealed connection with said first branch vessel stent graft, and said first connecting anchoring segment is retained at the cardiac-proximal end of said first opening (1121) of said first adjustment zone.

In one embodiment, said vessel is an aortic arch, and said positioning branch vessel or said first branch vessel is selected from the group consisting of left common carotid artery, innominate artery, and left subclavian artery.

In one embodiment, said vessel is an abdominal aorta, and said positioning branch vessel or said first branch vessel is selected from the group consisting of celiac trunk, left renal artery, right renal artery, and superior mesenteric artery.

In one embodiment, said vessel is an aortic root and ascending aorta, and said positioning branch vessel or said first branch vessel is selected from the group consisting of left coronary artery and right coronary artery.

In one embodiment, said vessel is any artery or vein comprising two or more branch vessels, and said positioning branch vessel or said first branch vessel is one of the branches of said vessel.

In one embodiment, said step (d) further comprises i) i) delivering a positioning branch vessel stent graft into said positioning branch vessel via said positioning assembly; said positioning assembly comprises a positioning sealing zone (111) and a positioning adjustment zone (112); said positioning sealing zone (111) comprises a positioning third opening (1112); said positioning adjustment zone (112) comprises a positioning first opening (1121), a positioning second opening (1122), a positioning first reinforcement structure (113), and a positioning second reinforcement structure (114); said positioning branch vessel stent graft comprises a positioning connecting anchoring segment, the diameter of said positioning connecting anchoring segment after being released is greater than said positioning first opening (1121), such that said positioning connecting anchoring segment is retained at the cardiac-proximal end of said first opening (1121) of said positioning adjustment zone; ii) releasing said positioning branch vessel stent graft, such that said positioning second opening aligns with the root of said positioning branch vessel, said positioning sealing zone (111) forms a planar sealed connection with said positioning branch vessel stent graft, and said positioning connecting anchoring segment is retained at the cardiac-proximal end of said first opening (1121) of said positioning adjustment zone (112).

In one embodiment, this invention provides method for implanting a stent graft system into a vessel, said vessel comprises a trunk, a positioning branch vessel, and a first branch vessel, wherein said stent graft system comprises: a) a stent graft, comprising: i) a positioning assembly; ii) a first branch vessel fenestration assembly (11), said first branch vessel fenestration assembly (11) comprises a first sealing zone (111) and a first adjustment zone (112); said first sealing zone (111) comprises a third opening (1112); said first adjustment zone (112) comprises a first opening (1121), a second opening (1122), a first reinforcement structure (113), and a second reinforcement structure (114); a first branch vessel stent graft, said first branch vessel stent graft is configured to be released within said first branch vessel fenestration assembly (11); said first branch vessel stent graft comprises a first connecting anchoring segment, the diameter of said first connecting anchoring segment after being released is greater than said first opening (1121) of said first adjustment zone, such that said first connecting anchoring segment is retained at the cardiac-proximal end of said first opening (1121) of said first adjustment zone; said method comprises the steps of: a) providing said stent graft system; b) delivering said stent graft into said trunk; c) aligning said positioning assembly with said positioning branch vessel; d) releasing said stent graft, such that said stent graft conforms to inner wall of said trunk and said positioning assembly aligns with the root of said positioning branch vessel; e) delivering said first branch vessel stent graft into said first branch vessel via said first branch vessel fenestration assembly (11); f) releasing said first branch vessel stent graft, such that said second opening aligns with the root of said first branch vessel, said first sealing zone (111) forms a sealed connection with said first branch vessel stent graft.

In one embodiment, said vessel, said positioning branch vessel, and said first branch vessel comprises a combination selected from the group consisting of: a) said vessel is the aortic arch, and said positioning branch vessel or said first branch vessel is selected from one of the left common carotid artery, the innominate artery, or the left subclavian artery; b) said vessel is the abdominal aorta, and said positioning branch vessel or said first branch vessel is selected from one of the celiac trunk, the left renal artery, the right renal artery, or the superior mesenteric artery; and c) said vessel is the aortic root and ascending aorta, and said positioning branch vessel or said first branch vessel is selected from one of the left coronary artery or the right coronary artery.

In one embodiment, said step (d) further comprises: i) delivering a positioning branch vessel stent graft into said positioning branch vessel via said positioning assembly; said positioning assembly comprises a positioning sealing zone (111) and a positioning adjustment zone (112); said positioning sealing zone (111) comprises a positioning third opening (1112); said positioning adjustment zone (112) comprises a positioning first opening (1121), a positioning second opening (1122), a positioning first reinforcement structure (113), and a positioning second reinforcement structure (114); said positioning branch vessel stent graft comprises a positioning connecting anchoring segment, diameter of said positioning connecting anchoring segment after being released is greater than said positioning first opening (1121), such that said positioning connecting anchoring segment is retained at the cardiac-proximal end of said positioning first opening (1121) of said positioning adjustment zone (112); and ii) releasing said positioning branch vessel stent graft, such that said positioning second opening aligns with the root of said positioning branch vessel, said positioning sealing zone (111) forms a sealed connection with said positioning branch vessel stent graft.

In one embodiment, said sealed connection is a planar sealed connection.

In one embodiment, said cardiac-proximal end of said first opening (1121) comprises any position close to the first opening.

In one embodiment, said cardiac-proximal end of the first opening comprises any position between the second opening and the first opening.

In one embodiment, said first connecting anchoring segment comprises a flange stent and a flange graft at its cardiac-proximal end; said cardiac-proximal end of the first opening is a position outside the first opening.

In one embodiment, said first branch vessel fenestration assembly (11) comprises one or more features selected from the group consisting of: a) first reinforcement structure (113) comprises a first radiopaque structure (115); b) said second reinforcement structure (114) comprises a second radiopaque structure (116); and c) said first adjustment zone (112) comprises a third radiopaque structure (117); wherein said step (e) further comprises the step of aligning said first branch vessel with said first branch vessel fenestration assembly (11) prior to step (f).

In one embodiment, said vessel further comprises a second branch vessel; said stent graft further comprises a second branch vessel fenestration assembly comprises a second sealing zone (111) and a second adjustment zone (112); said second sealing zone (111) comprises a third opening (1112); said second adjustment zone (112) comprises a first opening (1121), a second opening (1122), a first reinforcement structure (113), and a second reinforcement structure (114); said stent graft system further comprises a second branch vessel stent graft; said second branch vessel stent graft comprises a second connecting anchoring segment, diameter of said second connecting anchoring segment after being released is greater than said first opening (1121) of said second adjustment zone, such that said second connecting anchoring segment is retained at the cardiac-proximal end of said first opening (1121) of said second adjustment zone; said step (d) further comprises: i) delivering said second branch vessel stent graft into said second branch vessel via said second branch vessel fenestration assembly (11); and ii) releasing said second branch vessel stent graft, such that said second opening aligns with the root of said second branch vessel, said second sealing zone (111) forms a sealed connection with said second branch vessel stent graft, and said second connecting anchoring segment is retained at the cardiac-proximal end of said first opening (1121) of said second adjustment zone.

In one embodiment, said first branch vessel stent graft is delivered using a femoral or iliac artery approach.

In one embodiment, said step (f) comprises: i) releasing said first connecting anchoring segment; ii) adjusting position of said first branch vessel stent graft; and iii) releasing remaining segment of said first branch vessel stent graft.

In one embodiment, said stent graft system further comprises a radiopaque marker on a cardiac-distal end of said stent graft; and a straight stent graft with a radiopaque marker on a cardiac-proximal end; said step (d) further comprises: i) delivering said straight stent graft to cardiac-distal end of said stent graft; ii) aligning said radiopaque marker on said straight stent graft with said radiopaque marker on said stent graft; and releasing said straight stent graft to ensure said straight stent graft is expanded in an overlapping portion with said stent graft.

The primary technical problems addressed by the present invention are as follows:
1) The sealing zone (111) of the fenestration assembly of the present invention is formed by a flexible cylindrical or frustoconical graft structure. This flexible graft structure ensures sealing between the fenestration assembly and the branch vessel stent graft, effectively preventing endoleak and resolving the potential endoleak issues that may occur after implantation of branch vessel stent grafts in various fenestration designs;
2) The adjustment zone (112) of the fenestration assembly of the present invention allows the position and orientation of the second opening (1122) of the fenestration assembly to move and adjust within a certain range, thereby accommodating the anatomical variations that exist in most trunk and branch vessels;
3) The structure of the fenestration assembly of the present invention does not limit the access route for implanting the branch vessel stent graft system. All branch vessel stent grafts may be implanted via the femoral artery (lower access), thereby avoiding the postoperative cerebral complications that may result from plaque dislodgement from the inner wall of the branch vessels when techniques such as inner-tunnel or modular bridging require upper-access insertion. This also shortens the operative time and facilitates both physician operation and the broader adoption of the procedure.
4) The fenestrations of the present invention include reinforcement structures; which can isolate the vessel wall and the diseased region (false lumen, hematoma, etc.) after the stent graft is implanted into the vessel and can maintain the shape of the fenestration even when subjected to compression within the vessel. This ensures continued blood flow through the branch vessels and resolves the cerebral perfusion problems that may occur with other techniques that rely solely on recessed structures. In addition, the reinforced fenestration facilitates intraoperative superselection of the guidewire (“superselection” referring to the selective entry of a catheter or guidewire into the target branch vessel).
5) The fenestration assembly of the present invention includes radiopaque structures, which allow the position of the fenestration assembly to be clearly visualized under X-ray imaging during delivery and superselection. This facilitates intraoperative positioning of the stent graft, the superselection of the guidewire, and the positioning of the branch vessel stent grafts during the procedure.

The above features of the present invention are suitable for the repair and reconstruction of most vascular lesions (including dissecting aneurysms, true aneurysms, intramural hematomas, and multiple penetrating atherosclerotic ulcers) involving anatomical variations between the trunk and branch vessels. As described above, these include the anatomical variations between the aortic arch and its three supra-aortic branch vessels, as well as those between the abdominal aorta and the left and right renal arteries, the superior mesenteric artery, and the celiac trunk.

In one embodiment of the present invention, the cooperation among the various components of the stent graft system can effectively prevent endoleak that may otherwise occur after implantation of the branch vessel stent graft. In one embodiment, the connecting anchoring segment of the branch vessel stent graft has a post-release diameter greater than the first opening (1121) of the adjustment zone and is anchored at the cardiac-proximal end of the first opening (1121), which serves as the primary means of fixing the branch vessel stent graft. In one embodiment, the cardiac-proximal end of the first opening refers to any position around the first opening. In another embodiment, the cardiac-proximal end of the first opening refers to any position between the second opening and the first opening. The connecting anchoring segment of the branch vessel stent graft bears most of the fixation force, preventing separation between the branch vessel stent graft and the fenestration assembly and thereby preventing the occurrence of type III endoleak. When anatomical variations cause misalignment between the branch vessel and the fenestration assembly, the first reinforcement structure (113) and the second reinforcement structure (114) maintain the shapes of the first opening (1121) and the second opening (1122), facilitating guidewire superselection; at the same time, the second opening (1122) of the adjustment zone can automatically adjust its position during release of the branch vessel stent graft, allowing second opening (1122) to align automatically with the root of the branch vessel, thereby correcting misalignment and preventing kinking of the branch vessel stent graft. After the branch vessel stent graft is released and expanded, the sealing zone (111) forms a flexible cylindrical or frustoconical membrane structure. This flexible membrane forms a planar-to-planar seal with the branch vessel stent graft, effectively preventing endoleak.

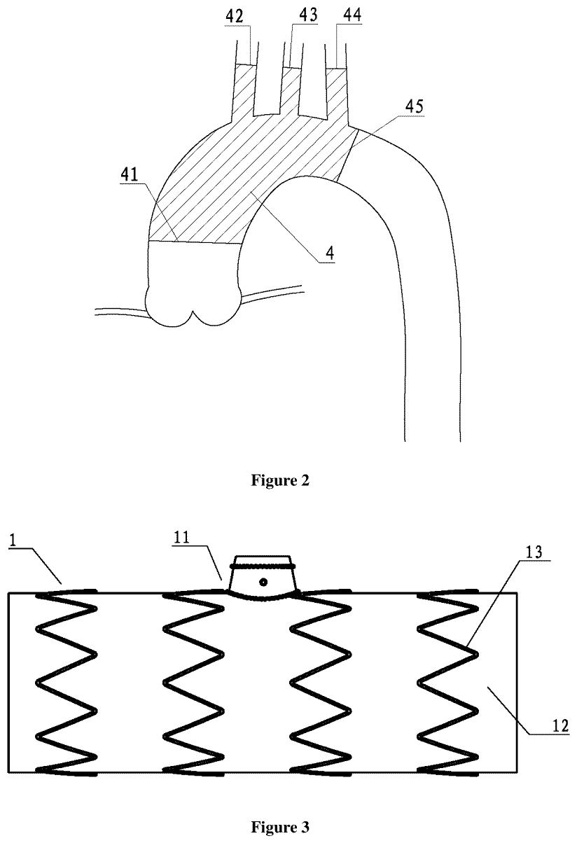

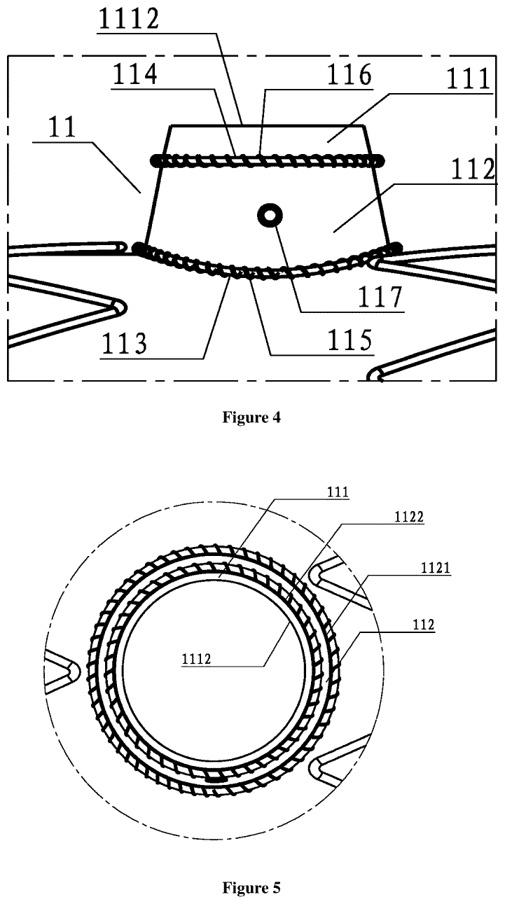

The present invention relates to a stent graft with a fenestration assembly for treating vascular diseases involving both the trunk and the branch vessels. The device of the present invention is a stent graft (1) with a fenestration assembly, which comprises a fenestration assembly (11), a cylindrical graft (12), and stent rings (13) (as shown in FIG. 3). The fenestration assembly (11) comprises a sealing zone (111), an adjustment zone (112), a first opening (1121), a second opening (1122), a third opening (1112), a first reinforcement structure (113), a second reinforcement structure (114), a first radiopaque structure (115), a second radiopaque structure (116), and a third radiopaque structure (117) (as shown in FIGS. 4 and 5). The third radiopaque structure (117) is an optional component.

The stent graft (1) with a fenestration assembly is used for treating vascular diseases involving both the trunk and the branch vessels. It is an assembly formed by combining multiple components through specific manufacturing processes and includes at least one fenestration assembly (11).

The fenestration assembly (11) is the connecting structure between the fenestrated stent graft (1) and the branch vessel stent grafts. It is an assembly formed by combining multiple components through specific manufacturing processes.

The cylindrical graft (12) is a layer of flexible cylindrical or frustoconical graft attached to the stent rings (13) by sewing, thermal bonding, or other methods, and is made of biocompatible materials.

The stent rings (13) are wave-shaped or mesh-shaped metallic wire frameworks.

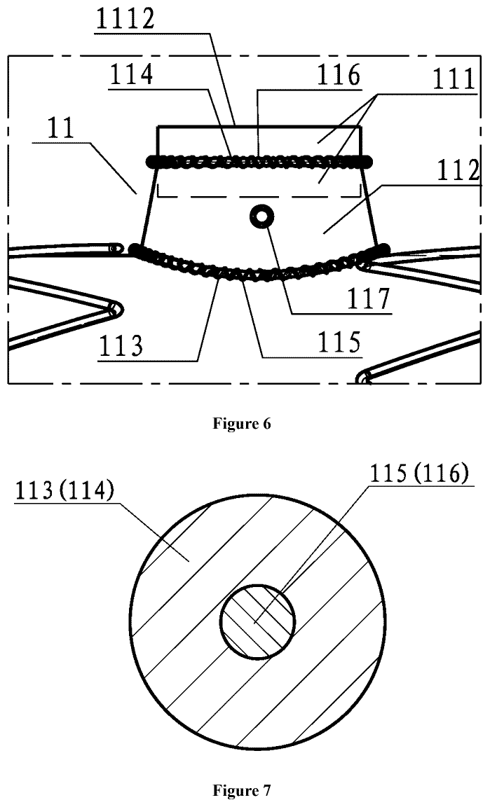

The sealing zone (111) is an upward-extending flexible cylindrical or frustoconical graft, which is attached to the second opening (1122) of the adjustment zone (112) by sewing, thermal bonding, or other methods, and is made of biocompatible materials. Optionally, the sealing zone (111) extends upward and downward simultaneously from the second opening (1122), as shown in FIG. 6.

The adjustment zone (112) is a flexible cylindrical or frustoconical graft attached to the cylindrical graft (12) by sewing, thermal bonding, or other methods, with a first opening (1121) and a second opening (1122) at its two ends, and is made of biocompatible materials.

The first opening (1121) is the opening of the adjustment zone (112) located on the side adjacent to the cylindrical graft (12). The first opening (1121) is circular or substantially circular in shape.

The second opening (1122) is the opening of the adjustment zone (112) located on the side distal to the cylindrical graft (12). The second opening (1122) is circular or substantially circular in shape, with a diameter or circumference less than that of the first opening (1121).

The third opening (1112) is the opening of the sealing zone (111) located on the side distal to the adjustment zone (112). The third opening (1112) is circular or substantially circular in shape, with a diameter or circumference less than or equal to that of the second opening (1122).

The first reinforcement structure (113) is an annular metal component attached to the first opening (1121) by sewing, thermal bonding, or other methods. The material of the first reinforcement structure (113), such as nitinol, is superelastic and provides a degree of radiopacity.

The second reinforcement structure (114) is an annular metal component attached to the second opening (1122) by sewing, thermal bonding, or other methods. The material of the second reinforcement structure (114), such as nitinol, is superelastic and provides a degree of radiopacity.

The first radiopaque structure (115) is a helical metal component attached to the first reinforcement structure (113), and its material, such as platinum alloys or gold, provides good radiopacity under X-ray imaging. In one embodiment, the helical metal component is attached to the first reinforcement structure (113) by winding. Optionally, the first radiopaque structure (115) is positioned at the center of the cross-section of the first reinforcement structure (113), as shown in FIG. 7.

The second radiopaque structure (116) is a helical metal component attached to the second reinforcement structure (114), and its material, such as platinum alloys or gold, provides good radiopacity under X-ray imaging. In one embodiment, the helical metal component is attached to the second reinforcement structure (114) by winding. Optionally, the second radiopaque structure (116) is positioned at the center of the cross-section of the second reinforcement structure (114), as shown in FIG. 7.

The third radiopaque structure (117) is a ring-shaped or disc-shaped metal component attached to the adjustment zone (112) by sewing, thermal bonding, or other methods, and its material, such as platinum alloys or gold, provides good radiopacity under X-ray imaging.

### Description of the General Functions of the Components:

The stent graft (1) with a fenestration assembly, which is used for treating vascular diseases involving branch vessels, reconstructs the branch vessels while reconstructing the trunk vessel, thereby maintaining patency of the branch vessels.

The fenestration assembly (11) serves as the structure that connects with the branch vessel stent graft. When the cylindrical graft (12) and the stent rings (13) are fixed in the trunk vessel, the fenestration assembly (11) can be combined with the branch vessel stent graft to accommodate different vascular anatomies.

The cylindrical graft (12) prevents blood from leaking to the outside of the stent graft and, together with the stent rings (13), functions to seal the intimal tear opening of the vessel or to occlude the aneurysmal cavity.

The stent rings (13) provide radial support, enabling the cylindrical graft (12) to be stably supported within the trunk vessel, and serve to expand the true lumen of the vessel while reducing the size of the false lumen.

The sealing zone (111) ensures sealing between the branch vessel stent graft and the fenestration assembly (11), thereby reducing endoleak.

The adjustment zone (112) enables the second opening (1122) to move and rotate within a certain range, thereby accommodating different anatomical configurations of the branch vessels.

The first opening (1121) is the opening through which blood flows into the adjustment zone (112).

The second opening (1122) is the opening through which blood flows into the sealing zone (111).

The third opening (1112) is the opening through which blood flows out of the sealing zone (111).

The first reinforcement structure (113) maintains the shape of the first opening (1121), ensures unobstructed blood flow through the branch vessels during and after the procedure, and facilitates intraoperative superselection.

The second reinforcement structure (114) maintains the shape of the second opening (1122), ensures unobstructed blood flow through the branch vessels during and after the procedure, and facilitates intraoperative superselection.

The first radiopaque structure (115) enhances the visibility of the shape of the first opening (1121) under intraoperative X-ray imaging, thereby facilitating intraoperative positioning of the stent graft (1) with a fenestration assembly, superselection of the branch vessel, and positioning of the branch vessel stent graft.

The second radiopaque structure (116) enhances the visibility of the shape of the second opening (1122) under intraoperative X-ray imaging, thereby facilitating intraoperative positioning of the stent graft (1) with a fenestration assembly, superselection of the branch vessel, and positioning of the branch vessel stent graft.

The third radiopaque structure (117) enhances the visibility of the position of the fenestration assembly (11) under intraoperative X-ray imaging, thereby facilitating intraoperative positioning of the stent graft (1) with a fenestration assembly, superselection of the branch vessel, and positioning of the branch vessel stent graft.

### Description of the Surgical Steps:

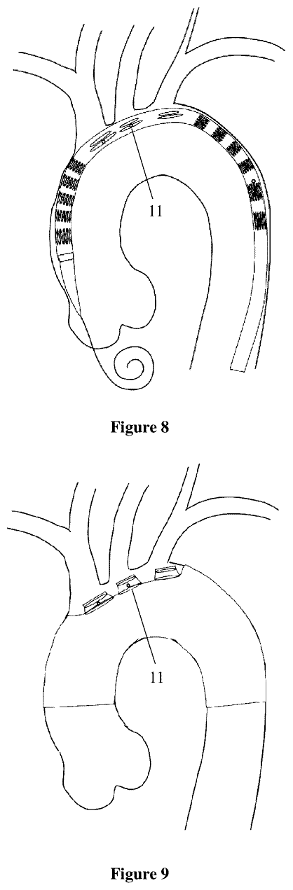

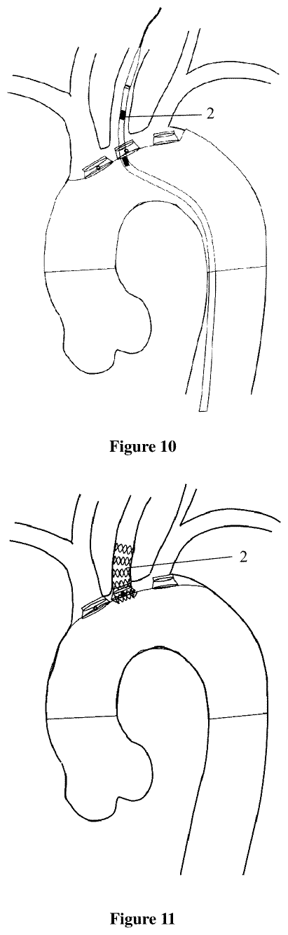

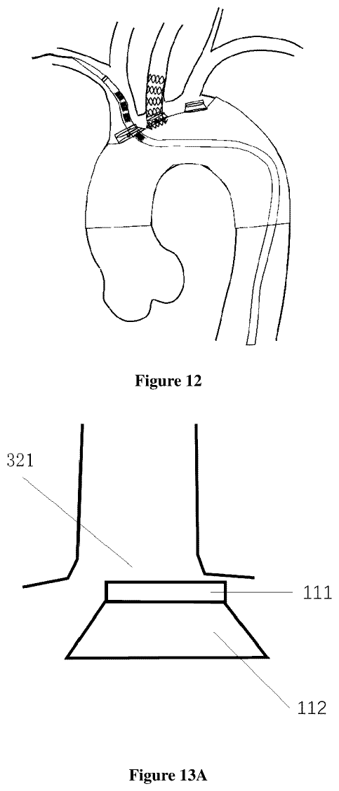

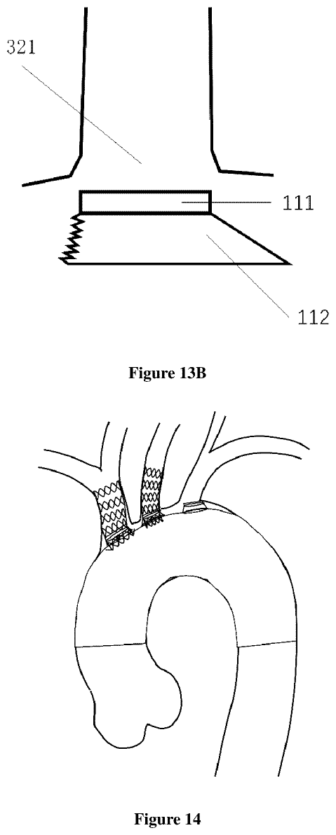

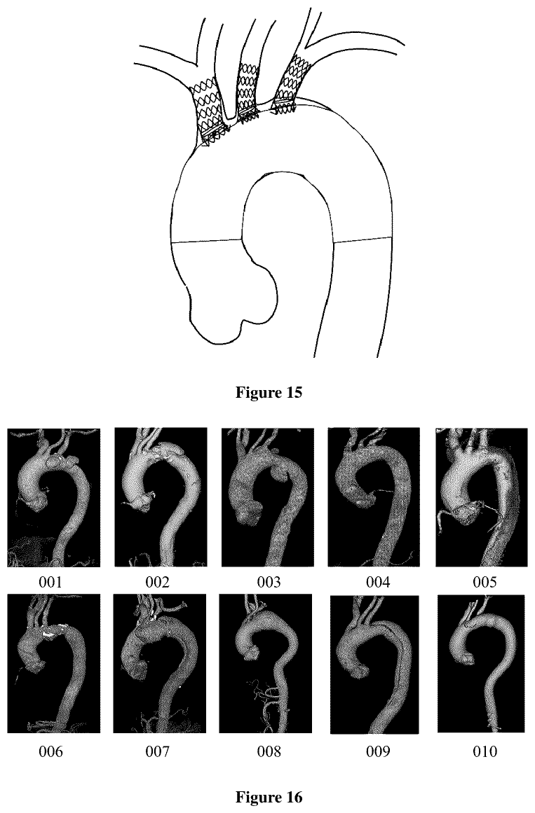

In the present invention, the procedure for treating vascular diseases involving both the trunk and the branch vessels can be simplified into the following five steps. For ease of explanation, the aortic arch region is selected here as the area requiring reconstruction.
i. As shown in FIG. 8, the fenestrated stent graft (1) is mounted on the delivery system. The fenestrated stent graft (1) comprises three fenestration assemblies (11), corresponding to the innominate artery, the left common carotid artery, and the left subclavian artery from left to right. By aligning the root of the left common carotid artery with the imaging structures (the first imaging structure (115), the second radiopaque structure (116), or the third radiopaque structure (117)) of the fenestration assembly (11) in the middle, the delivery system is advanced to the designated position.
ii. As shown in FIG. 9, the fenestrated stent graft (1) is released in the aorta. At the same time, the fenestrated stent graft (1) conforms to the inner wall of the aorta, and the fenestration assembly (11) in the middle is aligned with the root of the left common carotid artery.
iii. As shown in FIG. 10, the branch vessel stent graft (2) is mounted on the delivery system. By aligning the radiopaque structure (the first radiopaque structure (115)) of the fenestration assembly (11) in the middle with the radiopaque structure on the branch vessel stent graft (the first stent closest to the heart), the delivery system is advanced to the designated position.
iv. As shown in FIG. 11, the branch vessel stent graft (2) for the left common carotid artery is released. At the same time, the cardiac-proximal end of the branch vessel stent graft conforms to the sealing zone (111) of the fenestration assembly (11) of the fenestrated stent graft (1), while the cardiac-distal end conforms to the inner wall of the branch vessel.
v. As shown in FIG. 12, the branch vessel stent graft is mounted on the delivery system. By aligning the radiopaque structure (the first radiopaque structure (115)) of the leftmost fenestration assembly (11) with the radiopaque structure on the branch vessel stent graft (the first stent segment at the cardiac-proximal end), the delivery system is advanced to the designated position. As shown in FIGS. 13A and 13B, when the second opening (1122) of the leftmost fenestration assembly (11) is misaligned with the root (321) of the innominate artery, the adjustment zone (112) of the fenestration assembly (11) allows the second opening (1122) to move and rotate within a certain range, such that during guidewire superselection, advancement of the branch vessel stent graft system, or release of the branch vessel stent graft, the second opening (1122) can adjust its position and angle to align with the root (321) of the innominate artery.
vi. As shown in FIG. 14, the branch vessel stent graft for the innominate artery is released. At the same time, the cardiac-proximal end of the branch vessel stent graft conforms to the sealing zone (111) of the fenestration assembly (11) of the fenestrated stent graft (1), while the cardiac-distal end conforms to the inner wall of the branch vessel.
vii. As shown in FIG. 15, steps (v) and (vi) are repeated to implant the branch vessel stent graft for the left subclavian artery, thereby completing the vascular reconstruction of the aortic arch region.

For endovascular treatment of vascular diseases involving both the trunk and the branch vessels, techniques such as the in-situ fenestration technique, inner-tunnel techniques, and modular bridging techniques face major challenges, including anatomical variability of the branch vessels, endoleak, the requirement for implanting the branch vessel stent graft from the cardiac-distal end of the branch vessel, procedural complexity, and prolonged operative time. To address these issues, the present invention introduces the following innovations:
1) The sealing zone (111) of the fenestration assembly (11) ensures sealing between the fenestration assembly (11) and the branch vessel stent graft, effectively preventing endoleak. The sealing zone (111) provides sealing at a planar-level, which offers a substantially superior anti-leakage effect compared with sealing at linear-level. In one embodiment, the sealing zone (111) is a flexible cylindrical or frustoconical graft structure.
2) The position and angle of the openings of the fenestration assembly (11) of the present invention can be adjusted within a certain range, allowing accommodation of the anatomical variations of most branch vessels caused by individual differences. In addition, this significantly reduces the number of stent grafts (1) with fenestration assembly required, thereby lowering design and manufacturing complexity and reducing inventory requirements. In one embodiment, the adjustment zone (112) of the fenestration assembly (11) enables the position and angle of the second opening (1122) to be adjusted within a certain range, accommodating the anatomical variations present in most branch vessels.
3) The structure of the fenestration assembly (11) does not restrict the access route for the branch vessel stent graft system. The access route for implanting the branch vessel stent graft system may be selected based on the patient's actual vascular anatomy; that is, all branch vessel stent graft systems may be delivered via the femoral artery (lower access). Selecting lower access as the primary option not only greatly simplifies the procedure compared with upper access, but also reduces postoperative cerebral complications that may result from plaque dislodgement from the inner wall of the branch vessels during upper-access insertion. In addition, it substantially shortens the operative time.
4) The fenestration assembly (11) includes a first reinforcement structure (113) and a second reinforcement structure (114), which maintain the shape of the fenestration even when subjected to compression within the vessel, thereby preserving blood flow to the branch vessels and facilitating intraoperative guidewire superselection. This design also reduces the risk of intraoperative and postoperative stroke.
5) The fenestration assembly (11) includes a first radiopaque structure (115), a second radiopaque structure (116), and a third radiopaque structure (117). During delivery and superselection, these structures provide clear visualization of the fenestration under X-ray imaging, thereby facilitating intraoperative positioning of the fenestrated stent graft (1), guidewire superselection, and positioning of the branch vessel stent graft. As a result, the success rate of the procedure is improved.

### 1) Clinical CTA Evaluation

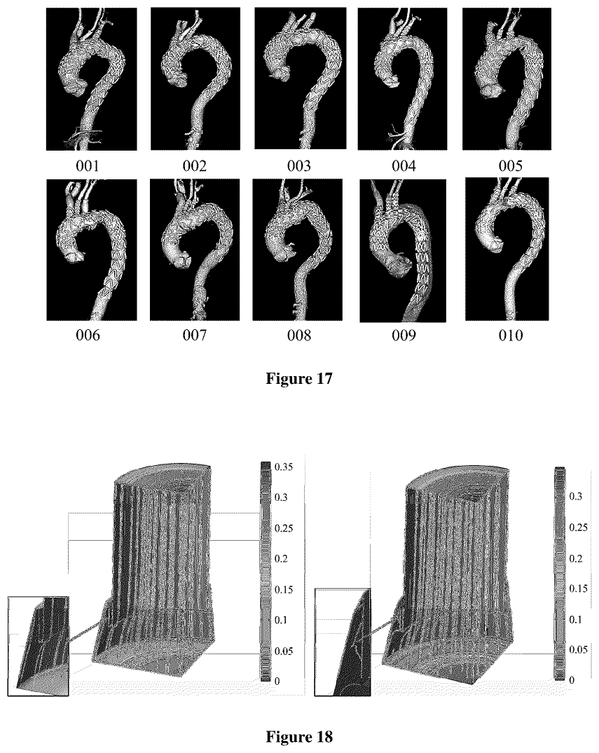

Using the stent graft system developed according to the present invention, ten clinical cases were performed. Postoperative CTA demonstrated that there was no endoleak between the branch vessel stent grafts and the fenestration assemblies, and that the branch vessel stent grafts were well aligned. The shortest procedure time was only 61 minutes, with an average of merely 112 minutes. Preoperative and postoperative CTA images are shown in FIGS. 16 and 17, respectively.

### 2) Finite Element Analysis

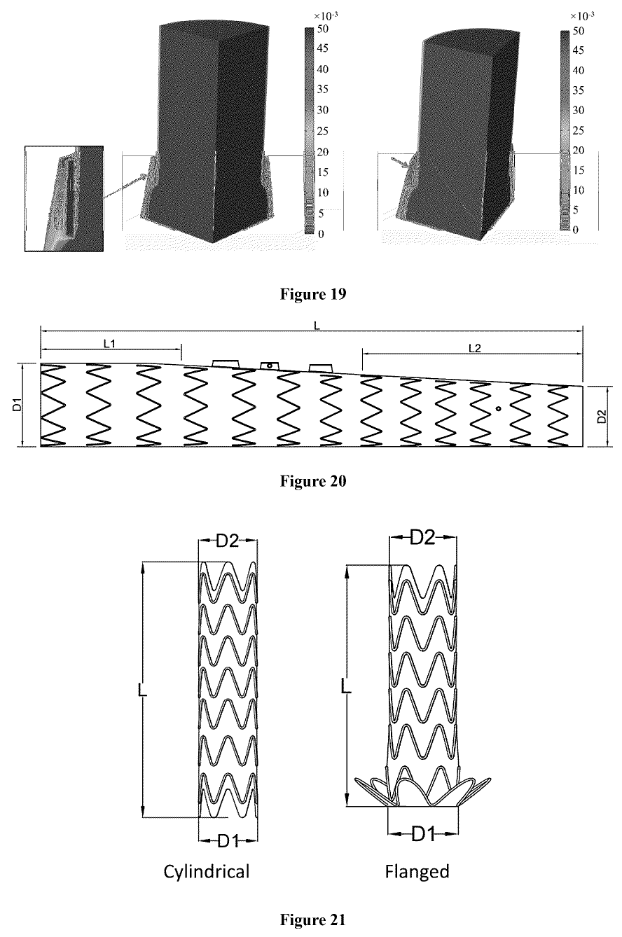

Finite element analysis demonstrated that, in the model without a sealing zone, endoleak occurred between the fenestration assembly and the branch vessel stent graft. While in the model with a sealing zone, no endoleak was observed in the corresponding region. Details are shown in FIGS. 18 and 19. FIG. 18 presents velocity contour plots and streamline diagrams: in the left panel, which corresponds to the model without a sealing zone, the streamlines between the fenestration assembly and the branch vessel stent graft are orderly, indicating the presence of endoleak; in the right panel, which corresponds to the model with a sealing zone, vortex flow is observed between the fenestration assembly and the branch vessel stent graft, indicating the absence of endoleak. FIG. 19 presents velocity contour plots: in the left panel, which corresponds to the model without a sealing zone, the flow velocity between the fenestration assembly and the branch vessel stent graft exceeds 0.05 m/s, indicating endoleak; in the right panel, which corresponds to the model with a sealing zone, the flow velocity between the fenestration assembly and the branch vessel stent graft is 0, indicating no endoleak.

### EXAMPLE

### Surgical Procedure for Stent System Implantation

### 1. Preoperative Arteriography

1.1) Use an introducer sheath to access the vessel at an appropriate site. It is recommended to use an introducer sheath with an inner diameter larger than the outer diameter of the delivery system. 1.2) Introduce a 0.035″ guidewire through the introducer sheath and advance said guidewire to beyond the proximal end of the target lesion. 1.3) Perform arteriography to confirm the location of the target lesion and the vessel dimensions, thereby determining the appropriate stent selection.

### 2. Preparation of the Stent System

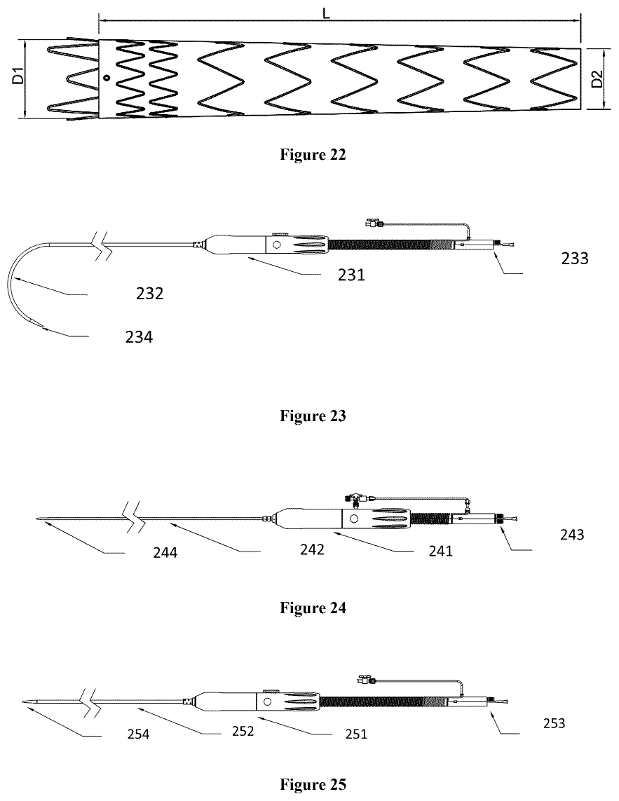

2.1) Verify that the main stent graft system (FIG. 20), the branch vessel stent graft system (FIG. 21), and the straight stent graft system (FIG. 22) are sterile and within their expiration dates, and check that the packaging is intact. 2.2) Remove the stent system with the intended specification from its package. Typical values of the various stents are listed in Table 1. 2.3) Inspect the product for any damage. Replace the product if damage is present; if the product is intact, continue the procedure. 2.4) Before inserting the stent system, tilt the delivery system so that the tapered tip points diagonally upward. Inject heparinized saline into the delivery system through a one-way valve until the heparinized saline flows out from the drainage groove on the side of the tapered tip, ensuring that the entire outer catheter is fully filled with the heparinized saline. 2.5) Inject heparinized saline through the connector at the tail end of the delivery system to flush the guidewire lumen of the delivery system. 2.6) Close the one-way valve of the delivery system. 2.7) Wipe the entire outer catheter surface with heparinized saline to activate the hydrophilic coating.

TABLE 1
Typical values of the various stents

| | L (mm) | L₁(mm) | L₂(mm) | D₁(mm) | D₂(mm) |
|---|---|---|---|---|---|
| main stent | 235 | 50-80 | 75-105 | 38-46 | 26-32 |
| graft | | | | | |
| branch vessel | 35-50 | / | / | 12-19 | 8-22 |
| stent graft | | | | | |
| straight stent | 160 | / | / | 24-36 | 18-30 |
| graft system | | | | | |

L₁: Length of the ascending aorta section;
L₂: Length of the descending aorta section
D₁: diameter at the cardiac-proximal end^{#};
D₂: diameter at the cardiac-distal end*
^{#}Cardiac-proximal end refers to the end of the stent that is closer to the heart
*Cardiac-distal end refers to the end of the stent that is away from the heart

### 3. Implantation of the Main Stent Graft

3.1) Introduce a 0.035″ guidewire through the introducer sheath and advance the distal end of the guidewire into the left ventricle. 3.2) Insert the Main Stent graft delivery system (FIG. 23) over the guidewire and advance the system along the guidewire to the patient's aortic arch. Note: Adjust the orientation of the delivery system handle (231) as needed so that the opening of the three-way valve with extension tubing (Luer) faces the direction opposite the curvature of the outer catheter (232) of the delivery system. This ensures that the major curvature of the outer catheter (232) of the delivery system aligns with the major curvature of the patient's aorta. 3.3) Confirm the stent position: (a) Ensure that the radiopaque marker for the left common carotid artery window of the main stent graft is aligned with the patient's left common carotid artery. (b) Observe the radiopaque markers at the head end of the main stent delivery system to ensure that the covered portion of the main stent graft does not cover the coronary ostia. Note: This step is critical. Failure to align the radiopaque markers accurately may result in incorrect stent positioning. 3.4) Stent deployment: (a) Slow-release: Hold the front handle with the left hand to fix its position and slowly rotate the rear handle with the right hand to gradually retract the outer catheter of the delivery system. (b) Fast-release: When the outer catheter has been retracted to the descending aorta segment, press and hold the fast-release button on the handle and pull the rear handle backward to fully deploy the stent. (c) Post-release: After full deployment of the stent in the descending aorta segment, unscrew and retract the post-release system (233) to fully expand the head end of the stent. Notes: Maintain the patient's blood pressure between 70-90 mmHg. Notes: During deployment, maintain forward support on the guidewire so that it remains close to the major curvature of the aorta. Notes: During deployment, continuously ensure that the radiopaque marker of the main stent graft for the left common carotid artery remains aligned with the proximal root of the patient's left common carotid artery. 3.5) Withdraw the delivery system: Hold the guidewire stationary and withdraw the entire delivery system from the body along the guidewire. Note: Withdraw the delivery system slowly to prevent the tapered tip (234) from catching on the stent. 3.6) Perform arteriography: Confirm smooth blood flow in the carotid artery. Then slowly withdraw the guidewire.

### 4. Implantation of the Branch Vessel Stent Grafts

It is recommended to implant the branch vessel stent grafts in the following sequence: the patient's left common carotid artery, innominate artery, and left subclavian artery. However, the physician may adjust the order according to the specific vascular anatomy and lesion characteristics. Using the left common carotid artery on the aortic arch as an example, the procedure is as follows:
4.1) Establish the branch guidewire channel: Advance an exchange catheter along the pre-positioned guidewire at the left common carotid artery opening of the main stent graft to the left common carotid artery fenestration on the main stent graft. Advance a loach-type guidewire along the exchange catheter to the left common carotid artery fenestration of the main stent graft, and super-selectively enter the patient's left common carotid artery, then withdraw the pre-positioned guidewire. After advancing the exchange catheter along the loach-type guidewire into the patient's left common carotid artery, withdraw the loach-type guidewire, insert a 0.035″ guidewire to an appropriate position within the left common carotid artery, and then withdraw the exchange catheter.
4.2) Implant the branch vessel stent graft: a) Advance the branch vessel stent graft delivery system along the 0.035″ guidewire into the patient's left common carotid artery. Position the system (FIG. 24) with the help of the arteriography imaging (the radiopaque marker of the branch vessel stent graft aligns with the radiopaque marker of the main stent graftfenestration). Stabilize the front handle (241) with the left hand and slowly rotate the rear handle (241) with the right hand to fully retract the outer catheter (242), thereby opening the cardiac-proximal end of the branch vessel stent graft. b) While fixing the position of the delivery system, unscrew and retract Post-Release System 1 (243) at the tail end of the delivery system at a uniform speed, allowing the branch vessel stent graft to fully expand. c) Unscrew and retract Post-Release System 2 (243) at the tail end of the delivery system, ensuring that the radiopaque marker at the cardiac-distal end of the branch stent graft separates from the inner core tube of the branch vessel stent delivery system. d) Withdraw the delivery system. Adjust the guidewire as needed. Retract the branch vessel stent delivery system so that the tapered tip (244) exits the stent. Reverse-rotate the rear handle to recapture the tapered tip in the descending aorta segment, then withdraw the entire delivery system from the body. e) Withdraw the 0.035″ guidewire. Note: Withdraw the delivery system slowly to prevent the tapered tip from catching on the stent. Implantation of the branch vessel stents for the innominate artery and left subclavian artery follows the same method above.

### 5. Implantation of the Straight Stent Graft

5.1) Introduce a 0.035″ guidewire and advance it to the aortic arch. 5.2) Insert the straight stent graft system (FIG. 25): Insert the straight covered delivery system over the guidewire and advance the system along the guidewire to the descending aorta. 5.3) Confirm the stent position: Ensure that the radiopaque marker at the cardiac-proximal end of the straight stent graft is aligned with the radiopaque marker at the cardiac-distal end of the main stent graft. 5.4) Stent deployment: a) Slow-release: Hold the front handle (251) with the left hand to fix its position and slowly rotate the rear handle (251) with the right hand to gradually retract the outer catheter (252) of the delivery system. b) Fast-release: When the overlapping portion between the straight stent graft and the main stent graft has fully expanded, press and hold the fast-release button on the handle and pull the rear handle backward to fully deploy the stent. c) Post-release: After full deployment of the stent in the descending aorta segment, unscrew and retract the post-release system (253) to fully expand the head end of the stent. 5.5) Withdraw the delivery system: Hold the guidewire stationary and withdraw the entire delivery system from the body along the guidewire. Note: Withdraw the delivery system slowly to prevent the tapered tip (254) from catching on the stent.

### 6. Completion of the Procedure

6.1) Evaluate the deployment performance of the stent. Perform angiography and observe the blood flow in the aorta and the branch arteries. If the branch vessel stent graft is not fully expanded, the physician may elect to perform balloon dilation (recommended pressure≤5 atm). 6.2) Withdraw all accessories, close the access site, and complete the procedure.

### REFERENCES

[1] Frost & Sullivan [symbol] [symbol] [symbol] [symbol] [Independent Market Research Report on the Aortic and Peripheral Vascular Interventional Device Industry.]. March 2019, https://www.frostchina.com/content/insight/detail?id=62f9d6c257d0761f000a3d2e.
[2] Czerny, Martin, et al. “EACTS/STS Guidelines for diagnosing and treating acute and chronic syndromes of the aortic organ.” European Journal of Cardio-Thoracic Surgery 65.2 (2024): ezad426.
[3] Sun, L. Z. “ [symbol] [Aortic Surgery].” Beijing: People's Medical Publishing House Co., Ltd. (2012): 159-162.
[4] Surgeon, T. “Chinese experts' consensus of standardized diagnosis and treatment for aortic dissection.” Chin. J. Thorac. Cardiovasc Surg 33.11 (2017): 641-654.

What is claimed is:

1. A method for implanting a stent graft system into a vessel, said vessel comprises a trunk, a positioning branch vessel, and a first branch vessel, wherein said stent graft system comprises:
a. a stent graft, comprising:
i. a positioning assembly;
ii. a first branch vessel fenestration assembly (11), said first branch vessel fenestration assembly (11) comprises a first sealing zone (111) and a first adjustment zone (112); said first sealing zone (111) comprises a third opening (1112);
said first adjustment zone (112) comprises a first opening (1121), a second opening (1122), a first reinforcement structure (113), and a second reinforcement structure (114);
b. a first branch vessel stent graft, said first branch vessel stent graft is configured to be released within said first branch vessel fenestration assembly (11); said first branch vessel stent graft comprises a first connecting anchoring segment, the diameter of said first connecting anchoring segment after being released is greater than said first opening (1121) of said first adjustment zone, such that said first connecting anchoring segment is retained at the cardiac-proximal end of said first opening (1121) of said first adjustment zone;
said method comprises the steps of:
i. providing said stent graft system;
ii. delivering said stent graft into said trunk;
iii. aligning said positioning assembly with said positioning branch vessel;
iv. releasing said stent graft, such that said stent graft conforms to inner wall of said trunk and said positioning assembly aligns with the root of said positioning branch vessel;
v. delivering said first branch vessel stent graft into said first branch vessel via said first branch vessel fenestration assembly (11);
vi. releasing said first branch vessel stent graft, such that said second opening aligns with the root of said first branch vessel, said first sealing zone (111) forms a sealed connection with said first branch vessel stent graft.

2. The method of claim 1, wherein said vessel, said positioning branch vessel, and said first branch vessel are selected from the following combinations:
i. said vessel is the aortic arch, and said positioning branch vessel or said first branch vessel is selected from one of the left common carotid artery, the innominate artery, or the left subclavian artery;
ii. said vessel is the abdominal aorta, and said positioning branch vessel or said first branch vessel is selected from one of the celiac trunk, the left renal artery, the right renal artery, or the superior mesenteric artery; or
iii. said vessel is the aortic root and ascending aorta, and said positioning branch vessel or said first branch vessel is selected from one of the left coronary artery or the right coronary artery.

3. The method of claim 1, wherein said step (d) further comprises:
i. delivering a positioning branch vessel stent graft into said positioning branch vessel via said positioning assembly; said positioning assembly comprises a positioning sealing zone (111) and a positioning adjustment zone (112); said positioning sealing zone (111) comprises a positioning third opening (1112); said positioning adjustment zone (112) comprises a positioning first opening (1121), a positioning second opening (1122), a positioning first reinforcement structure (113), and a positioning second reinforcement structure (114); said positioning branch vessel stent graft comprises a positioning connecting anchoring segment, diameter of said positioning connecting anchoring segment after being released is greater than said positioning first opening (1121), such that said positioning connecting anchoring segment is retained at the cardiac-proximal end of said positioning first opening (1121) of said positioning adjustment zone (112);
ii. releasing said positioning branch vessel stent graft, such that said positioning second opening aligns with the root of said positioning branch vessel, said positioning sealing zone (111) forms a sealed connection with said positioning branch vessel stent graft.

4. The method of claim 1, wherein said sealed connection of step (f) is a planar sealed connection.

5. The method of claim 1, wherein said cardiac-proximal end of said first opening (1121) is any position close to the first opening.

6. The method of claim 1, wherein said cardiac-proximal end of the first opening is any position between the second opening and the first opening.

7. The method of claim 1, wherein said first connecting anchoring segment has a cardiac-proximal end comprising a flange stent and a flange graft; said cardiac-proximal end of the first opening is a position outside the first opening.

8. The method of claim 1, wherein said first branch vessel fenestration assembly (11) comprises one or more features selected from the group consisting of:
i. first reinforcement structure (113) comprises a first radiopaque structure (115);
ii. said second reinforcement structure (114) comprises a second radiopaque structure (116); and
iii. said first adjustment zone (112) comprises a third radiopaque structure (117);
wherein said step (e) further comprises the step of aligning said first branch vessel with said first branch vessel fenestration assembly (11) prior to step (f).

9. The method of claim 1, wherein:
a. said vessel further comprises a second branch vessel;
b. said stent graft further comprises a second branch vessel fenestration assembly comprises a second sealing zone (111) and a second adjustment zone (112); said second sealing zone (111) comprises a third opening (1112); said second adjustment zone (112) comprises a first opening (1121), a second opening (1122), a first reinforcement structure (113), and a second reinforcement structure (114);
c. said stent graft system further comprises a second branch vessel stent graft; said second branch vessel stent graft comprises a second connecting anchoring segment, diameter of said second connecting anchoring segment after being released is greater than said first opening (1121) of said second adjustment zone, such that said second connecting anchoring segment is retained at the cardiac-proximal end of said first opening (1121) of said second adjustment zone;
said step (d) further comprises:
i. delivering said second branch vessel stent graft into said second branch vessel via said second branch vessel fenestration assembly (11);
ii. releasing said second branch vessel stent graft, such that said second opening aligns with the root of said second branch vessel, said second sealing zone (111) forms a sealed connection with said second branch vessel stent graft, and said second connecting anchoring segment is retained at the cardiac-proximal end of said first opening (1121) of said second adjustment zone.

10. The method of claim 1, wherein said stent graft system further comprises a radiopaque marker on a cardiac-distal end of said stent graft; and a straight stent graft with a radiopaque marker on a cardiac-proximal end; said step (d) further comprises:
i. delivering said straight stent graft to cardiac-distal end of said stent graft;
ii. aligning said radiopaque marker on said straight stent graft with said radiopaque marker on said stent graft; and
iii. releasing said straight stent graft to ensure said straight stent graft is expanded in an overlapping portion with said stent graft.

11. The method of claim 1, wherein said first branch vessel stent graft is delivered using a femoral or iliac artery approach.

12. The method of claim 1, wherein said step (f) comprises:
i. releasing said first connecting anchoring segment;
ii. adjusting position of said first branch vessel stent graft; and
iii. releasing remaining segment of said first branch vessel stent graft.
